(19) United States
(12) Patent Application Publication
(10) Pub. No.: US 2009/0069786 A1
(43) Pub. Date: Mar. 12, 2009

(54) Medical apparatus and method for facilitating the management of long term tunneled conduits

(75) Inventors: Thomas M. Vesely, Saint Louis, MO (US); Christopher H. Porter, Woodinville, WA (US); Claude A. Vidal, Santa Barbara, CA (US); Russell J. Redmond, Goleta, CA (US); Byron L. Moran, Santa Barbara, CA (US); Paul Kaluzniak, St. Paul, MN (US); Abram D. Janis, Valencia, CA (US)

Correspondence Address: ARTHUR FREILICH;FREILICH, HORNBAKER & ROSEN, 20555 DEVONSHIRE ST. #372, CHATSWORTH, CA, 91311, US

(73) Assignee: Medical Research Products-B, Inc.

(21) Appl. No.: 12/286,821

(22) Filed: Oct. 1, 2008

Related U.S. Application Data
(63) Continuation-in-part of application No. 11/708,445, filed on Feb. 20, 2007.
(60) Provisional application No. 60/818,768, filed on Jul. 5, 2006.
(60) Provisional application No. 60/998,848, filed on Oct. 12, 2007.

(51) Int. Cl. A61M 5/32

(52) U.S. Cl. 604/500; 604/175

(57) ABSTRACT

A method and apparatus for facilitating the management of indwelling conduits to avoid and/or treat infections associated with long term implantation. A sleeve carrying a layer of porous material on its outer surface is percutaneous implanted to place the layer of porous material just under the patient's outer skin in contact with the patient's dermis where it functions to promote soft tissue ingrowth. A catheter passes through the sleeve passageway and along an interior body path to an interior destination site, e.g., an opening into a vein, i.e., venotomy site. Within about 3-6 weeks after implantation, the patient's dermal tissue integrates sufficiently with the sleeve porous material to physically anchor the sleeve and create an infection resistant barrier. Further, a tunnel, characterized by an epithelialized capsule, typically forms around the catheter along the interior body path extending to the interior destination site. After sufficient integration of the patient's soft tissue into the porous layer, the sleeve passageway can be used to pass various procedural tools to the tunnel and interior destination site without disturbing the tissue integrating barrier.

### RELATED APPLICATIONS

This application is a Continuation-In-Part of U.S. application Ser. No. 11/708,445 filed on Feb. 20, 2007 which claims priority based on U.S. Provisional Application 60/818,768 filed on Jul. 5, 2006. This application also claims priority based on U.S. Provisional Application 60/998,848 filed on Oct. 12, 2007. The aforementioned applications are, by reference, incorporated herein.

### FIELD OF THE INVENTION

This invention relates generally to medical technology and more particularly to a method and apparatus for facilitating the management of indwelling conduits, e.g., catheters, to avoid and/or treat infections which often occur as a consequence of the conduit being implanted for long periods of time, e.g., greater than 30 days. Embodiments of the invention are useful in a variety of applications which employ percutaneous conduits, e.g., in hemodialysis procedures where a percutaneous catheter provides fluid access to a patients central venous system and/or nerve stimulation procedures in which a percutaneous cable provides access to an implanted electric device.

### BACKGROUND OF THE INVENTION

For a variety of medical procedures, a conduit, most typically a catheter, is implanted in a patient so that it extends through a skin incision and along an interior path to an interior destination site; e.g., a venotomy site. When a catheter remains implanted over a long period, e.g., greater than 30 days, infections frequently occur, generally at the incision site where the catheter enters the body or along the interior path, or tunnel, or at the destination site, e.g., the entrance point to a vein. These problems are widely discussed in the literature; e.g., see “Long-Term Management Of The Tunneled Venous Catheter” by Liangos et al, Seminars In Dialysis 19(2) 158-164.

The aforementioned U.S. application Ser. No. 11/708,445 describes an apparatus and method of use for percutaneously implanting an elongate conduit, e.g., a catheter or cable, in a patient's body in a manner which allows the conduit to be easily positioned, repositioned, and replaced. Briefly, the exemplary apparatus described in said application Ser. No. 11/708,445 includes an elongate sleeve designed to be implanted for very long periods, e.g., several months to years. The sleeve comprises a wall surrounding an interior elongate passageway which extends from a sleeve proximal end to a sleeve distal end. The sleeve is intended to be percutaneously implanted through an incision in the patient's skin so that the sleeve distal end resides beneath the skin, i.e., subcutaneously, and the sleeve proximal end resides outside the skin. The sleeve outer peripheral surface carries a layer of porous material, e.g., a biocompatible mesh, intended to be placed under the patient's outer skin layer in contact with the dermis to promote tissue ingrowth for anchoring the sleeve and forming an infection resistant barrier. The sleeve passageway is dimensioned to snugly accommodate the outer surface of a conduit (which will hereinafter be assumed to be a catheter unless otherwise stated) while permitting the catheter to slide and rotate in the passageway relative to the sleeve. A sealing device within the sleeve extends around the catheter, e.g., near the sleeve proximal end, to prevent infectious material from migrating into the patient's body along the catheter outer surface.

A locking member mounted at the sleeve's proximal end is configured so it can be readily manipulated by a physician to selectively define either a first, or unlocked, state, and a second, or locked, state. In the unlocked state, the catheter is able to slide and/or rotate relative to the sleeve passageway. In the locked state, a friction force is applied to the catheter to prevent relative movement between the catheter and the sleeve.

### SUMMARY OF THE INVENTION

The present invention is directed to a method and apparatus for facilitating the management of indwelling conduits to avoid and/or treat infections associated with long term implantation.

In accordance with the present invention, a sleeve, for example of the type described in said application Ser. No. 11/708,445, carrying a layer of porous material on its outer surface is percutaneously implanted to place the layer of porous material just under the patient's skin in contact with the patient's dermis where it functions to promote soft tissue ingrowth. A catheter passes through the sleeve passageway and along an interior body path to an interior destination site, e.g., an opening into a vein, i.e., venotomy site. Within about 3-6 weeks after implantation, the patient's dermal tissue integrates sufficiently with the sleeve porous material to physically anchor the sleeve and create an infection resistant barrier. Further, a tunnel, characterized by an epithelialized capsule, typically forms around the catheter along the interior body path extending to the interior destination site. After sufficient integration of the patient's soft tissue into the porous layer, the sleeve passageway can be used, in accordance with the invention, to pass various procedural tools to the tunnel and interior destination site without disturbing the tissue integrating barrier. The procedural tools can be used for a variety of purposes. For example, the tools can include a balloon catheter for treating thrombosis or stenosis and for implanting a vascular stent, an angiography catheter for introducing contrast media, an infusion catheter for introducing medication, etc.

To prevent outward blood flow when inserting tools into the sleeve, it is preferable to provide a hemostatic valve connected in series with the sleeve passageway. In an exemplary embodiment, the hemostatic valve can be mounted within the sleeve passageway. In a preferred embodiment, a portal device is provided having at least one lumen whose distal end is adapted for detachable coupling to the proximal end of the sleeve. The portal device includes an entrance port at the lumen proximal end. An adjustable hemostatic valve, e.g., Touhy-Borst, is preferably detachably coupled to the portal device entrance port. The portal device preferably also incorporates a closure device, or cap, for temporarily closing the lumen to seal the passageway and tunnel.

It is generally known that an epithelialized capsule, or tunnel, typically forms around a long term hemodialysis catheter. Inasmuch as preservation of the tunnel and venotomy site is often considered important to the long term success of a procedure, it is frequently advantageous to remove an implanted catheter for a short period, e.g., 24 to 72 hours, to facilitate the treatment of an infection in the tunnel or near the venotomy site or in the bloodstream. Embodiments of the invention allow for a catheter to be withdrawn from the sleeve to allow the infection to be treated by infusing antibiotics or other agents into the tunnel, and for subsequently reinserting a new catheter through the sleeve passageway and tunnel. The sleeve and tunnel will often guide the catheter to the venotomy site and into the blood vessel to reestablish vascular access without requiring a major interventional procedure. Moreover, the foregoing can be performed without disrupting the tissue integrity barrier formed at the sleeve porous layer.

### BRIEF DESCRIPTION OF THE FIGURES

FIG. 1 is a schematic representation generally depicting a catheter assembly used in accordance with the invention for percutaneously implanting a catheter for an exemplary hemodialysis application;

FIG. 2 is an isometric view of a preferred catheter assembly;

FIG. 3 is an exploded view of the assembly of FIG. 2 showing a catheter in phantom together with a protective sheath, an anchor, a sleeve carrying a layer of porous material, an annular seal, and a locking member;

FIG. 4A is a sectional view taken substantially along the plane of 4A-4A of FIG. 2;

FIG. 4B is a sectional view taken substantially along the plane 4B-4B of FIG. 4A showing the locking member in its unlocked state;

FIG. 4C is a sectional view similar to FIG. 4B showing the locking member in its locked state clamped by suture or wire;

FIGS. 4D and 4E show exemplary spring clips which can be alternatively used for clamping the locking member in its locked state;

FIG. 5 is a plan view of the protective sheath of FIG. 3;

FIG. 6 is a sectional view taken substantially along the plane 6-6 of FIG. 5 particularly showing a perforated score line;

FIG. 7 is an isometric view of a preferred portal device in accordance with the present invention;

FIG. 8 is a side view of the portal device of FIG. 7;

FIG. 9 is a plan view of a commercially available Touhy-Borst hemostatic valve assembly which can be used in combination with the portal device of FIG. 7; and

FIG. 10 shows an exemplary procedural tool (i.e., angioplasty balloon) inserted through the hemostatic valve, portal device, and sleeve for accessing an interior body site.

### DETAILED DESCRIPTION

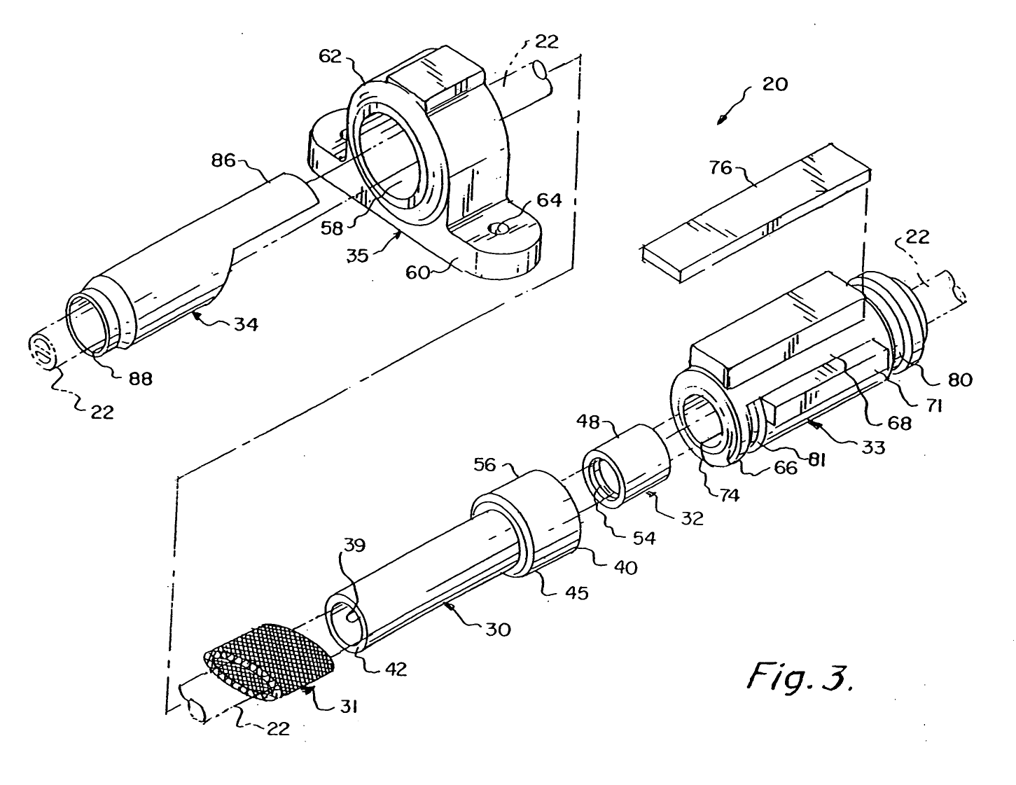

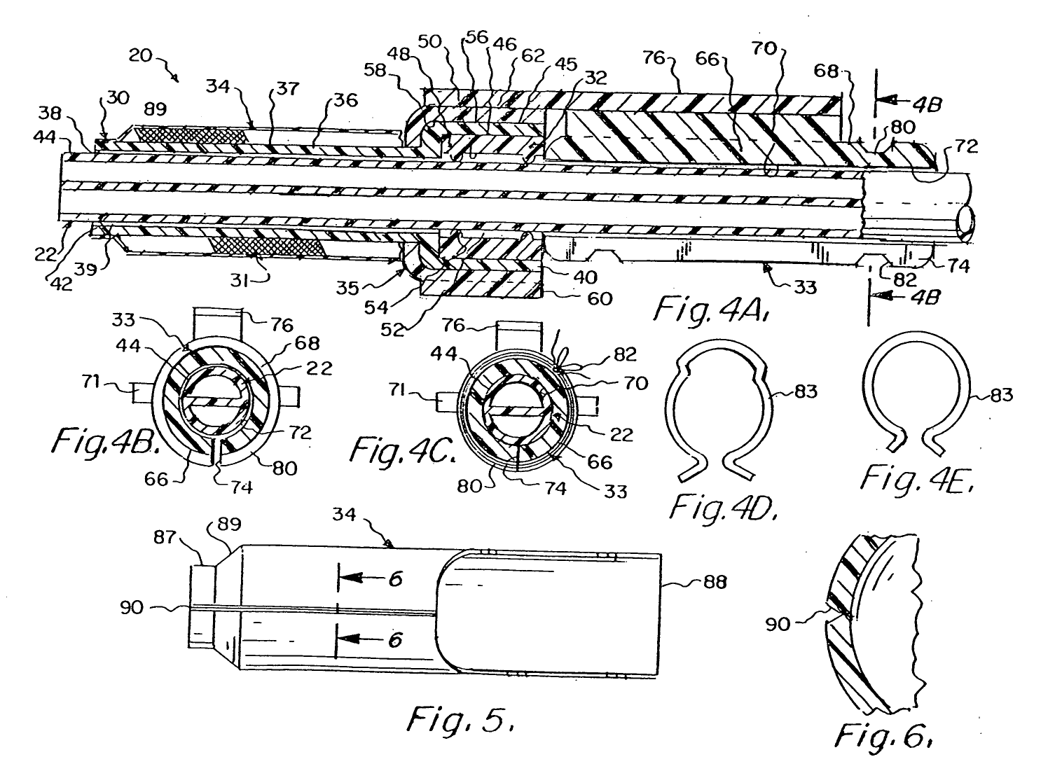

Various medical regimens relating, for example, to hemodialysis drug infusion, plasmapheresis, etc., use a percutaneously implanted conduit for conveying fluid and/or electric signals to/from an interior body site. The present invention is directed to a method and apparatus for facilitating the management of a percutaneous conduit (e.g., catheter) intended for use over a long term, e.g., greater than 30 days. A preferred embodiment of the present invention utilizes apparatus of the type exemplified by said U.S. application Ser. No. 11/708,445. For convenience, FIGS. 1-6 of this application duplicate the corresponding figures of said application Ser. No. 11/708,445 but it should be understood that the present invention is not restricted to the use of this particular apparatus.

FIG. 1 schematically depicts an assembly 20 for percutaneously implanting a catheter 22 through an incision 24 in a patient 26 undergoing an exemplary hemodialysis procedure. In such a procedure, a dual lumen catheter 22 is typically used with the two lumens being respectively coupled to separate exterior flow couplers 28 and 29.

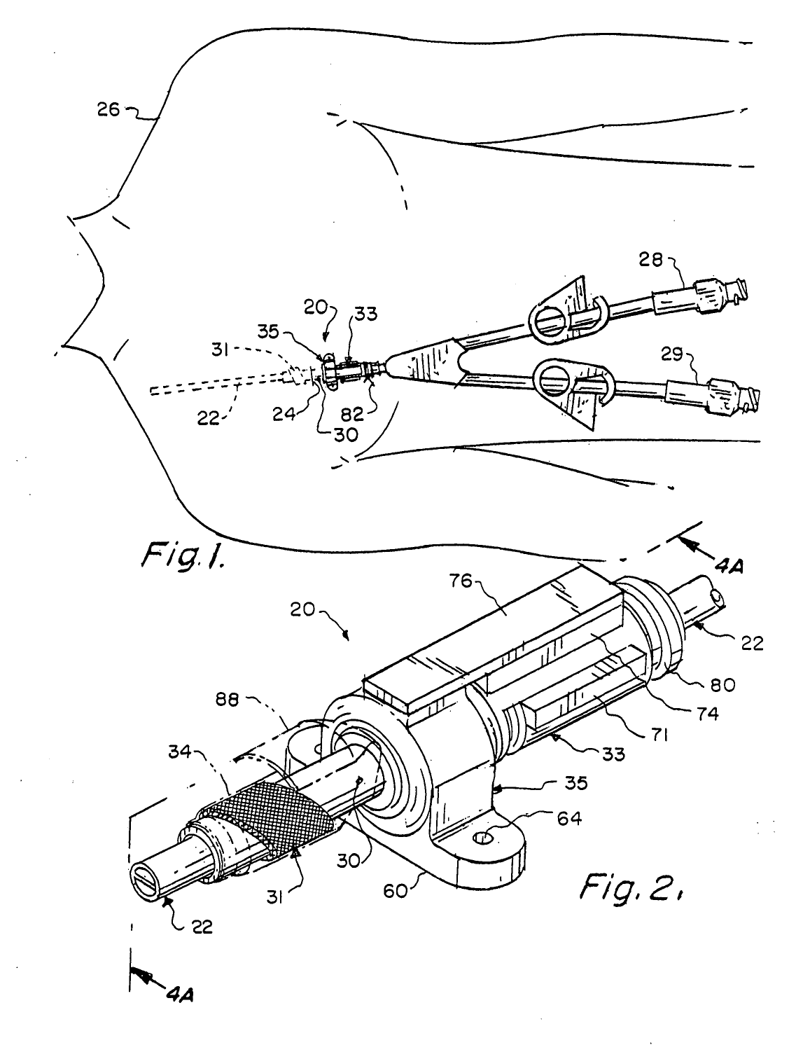

Attention is now directed to FIGS. 2-4 which show the primary elements of the assembly 20 including sleeve 30 carrying a porous layer 31, a sealing device 32, and a locking member 33. The assembly 20 also preferably includes an optional removable protective sheath 34 and an anchor 35 for anchoring the assembly 20 to a patient's outer skin surface. The sleeve 30 preferably comprises a substantially rigid tubular member formed of biocompatible material, e.g., titanium. The sleeve 30 includes a peripheral wall 36 (FIG. 4) having an outer surface 37 and an inner surface 38. The inner surface 38 surrounds an interior passageway 39 extending axially from a sleeve first, or proximal, end 40 to a sleeve second, or distal, end 42.

The sleeve 30 is shown mounted on a catheter 22 extending axially through the passageway 39. The catheter outer surface 44 and passageway wall surface 38 are closely dimensioned but with sufficient clearance therebetween to enable the catheter to slide axially and rotate in the passageway 39. The sleeve 30 proximal end 40 is preferably enlarged at 45 to form an interior recess 46 for accommodating the sealing device 32. The sealing device 32 preferably comprises an annular member 48 formed of a soft flexible material, e.g., silicone. The seal member 48 defines an inner peripheral surface 50 surrounding an interior bore 52 which is contiguous with sleeve passageway 39. At least one flexible annular nib 54 extends radially into the bore 52 for contacting and sealing against the catheter outer surface 44.

The enlarged sleeve end 45 has an outer peripheral surface 56 dimensioned to closely fit into bore 58 of anchor 35. The anchor 35 comprises a base portion 60 supporting a ferrule portion 62 which defines the bore 58. The anchor base portion 62 is provided with holes 64 to facilitate the suturing of anchor 35 to the patient's skin.

The locking member 33 preferably comprises a split ring formed of soft flexible material, e.g., silicone. More particularly, the locking member 33 is comprised of a peripheral wall 66 having an outer surface 68 and an inner surface 70 surrounding an interior bore 72. The wall 66 is longitudinally split at 74. The wall outer surface 68 is preferably provided with one or more strap pads 71 for securing the locking member 33 to the anchor 35 and/or sleeve 30 using one or more straps 76. The locking member outer wall surface is provided with a proximal annular groove 80 for accommodating suture thread or an appropriately shaped spring clip which can be used by a physician to compress the locking member 33 around the catheter 22. Preferably, a distal annular groove 81 is also provided.

The locking member 33 is configured so that in its natural unlocked state (FIG. 4B), the interior bore 72 is sufficiently large to permit the catheter 22 to slide axially and rotate in the bore 72 and through the sleeve passageway 39. The physician can compress the locking member wall 66 around the catheter to frictionally engage the locking member inner surface 70 against the catheter outer surface 44 to thus lock the catheter outer surface 44 to the sleeve 30 to prevent any relative movement therebetween. This locked state can be maintained by tying suture thread 82 around the locking member wall in grooves 80, 81. Of course, the thread 82 can be readily cut when it is desired to release the locked state to allow the catheter to be repositioned and/or replaced. FIGS. 4D and 4E depict exemplary spring clips 83 which can be alternatively placed in the grooves 80, 81 in lieu of thread 82 for clamping the locking member in its locked state.

The layer of porous material 31, e.g., titanium mesh, as described in U.S. application Ser. No. 10/821,383, is mounted around the outer surface 37 of sleeve 30, close to the sleeve distal end 42. In use, it is intended that the sleeve distal end be inserted through an incision 24 in the patient's skin to position the porous layer 31 just below the patient's epidermal skin layer 84 and in contact with the patient's dermal layer 85. Note that the porous layer 31 is preferably oriented diagonally with respect to the axis of sleeve 30 to better conform to the patient's skin contour. This orientation optimizes contact between the porous layer 31 and the patient's subcutaneous tissue to promote, over time, ingrowth into the porous layer. This tissue ingrowth acts to firmly anchor the sleeve in place and to form an infection resistant barrier around sleeve 30. This barrier may be enhanced by incorporating antimicrobial, bioactive, and/or anti-inflammatory constituents into the porous layer 31. For example, silver containing compounds and/or antibiotic eluting and/or growth factor coatings can be used as antimicrobial agents and steroids can be used as anti-inflammatory agents.

The aforementioned protective sheath 34 is formed of thin flexible tubular material (e.g., 0.010″ wall FEP tubing) and is intended to be mounted around sleeve 30 and porous layer 31 prior to use to avoid injuring the patient's tissue when the sleeve distal end 42 is inserted through the incision 24. As described in said U.S. application Ser. No. 11/708,445, the sheath 34 is removed from the sleeve 30 by the physician after the sleeve and porous layer have been inserted through the incision.

More particularly, the sheath 34 is preferably configured as a substantially tubular, e.g., cylindrical, body 86 having a distal collar 87 and a proximal elongate pull tab 88. An outwardly tapering section 89 extends from the collar 87 to the main body portion 86. Note that the collar 87 and distal portion of section 89 have a diameter smaller than that of the porous layer 31. For example only, the sleeve 30 may have an outer diameter of 0.250 inches, the porous layer 31 an outer diameter of 0.310 inches and the collar 87 an inner diameter of 0.193 inches. An axially oriented score, or perforated line 90 is preformed through the collar 87, the tapering section 89 and the body portion 86 to facilitate the physician peeling the sheath 34 from the sleeve 30. Note in FIG. 4A that the sheath fits tightly around the periphery of sleeve 30 and porous layer 31 and that the tapering section 89 is positioned distally of the porous layer 31. In use, the physician is able to readily peel the sheath from the sleeve with one hand by rolling, or winding, the elongate tab to pull the sheath axially in a proximal direction. Peeling occurs because as the sheath is pulled proximally, the tapering section 89 and collar 87 have to move past the larger diameter porous layer 31 which action causes the sheath to tear along score line 90 allowing it to be easily stripped from the sleeve 30.

In the preferred catheter assembly illustrated in FIGS. 2-4A, the sleeve 30 comprises a rigid titanium tube characterized as follows:

| | |
|---|---|
| overall length | 1.135 inches |
| proximal end 45 length | .250 inches |
| passageway 39 ID | .200 inches |
| end 45 ID | .313 inches |
| sleeve 30 wall thickness | .025 inches |
| porous material 31 OD | .304 inches |
| nib 54 ID | .170 inches |

In an alternative embodiment, the sleeve can be similarly dimensioned but instead of being formed of a rigid material such as titanium, can be formed of a flexible material such as silicone. In such an embodiment, the annular sealing nibs 54 can be integrally formed with the sleeve.

As previously mentioned the apparatus of FIGS. 1-6 enables a physician to replace an implanted catheter by unlocking the locking member 33 and sliding the implanted catheter proximally through the sleeve 30.

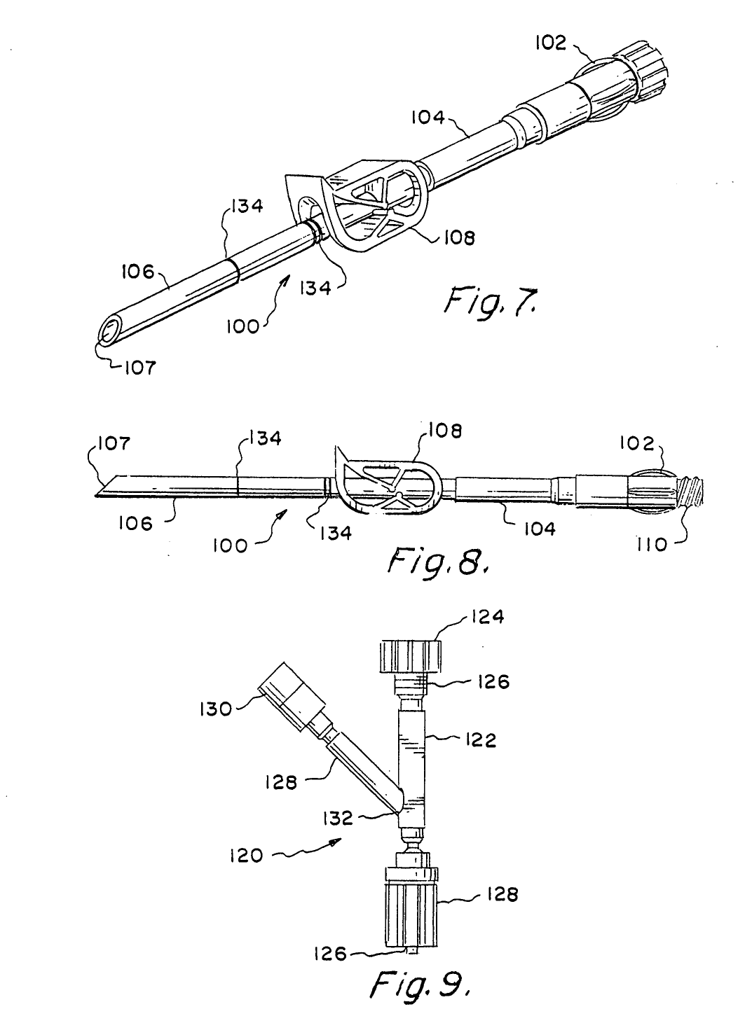

Attention is now directed to FIGS. 7 and 8 which illustrate a preferred portal device 100 in accordance with the present invention. The portal device 100 is intended to be used in combination with the aforedescribed catheter assembly 20 to allow various procedural tools, e.g., balloon and angiographic catheters, access to the interior body path and to allow the selected infusion of medication for treating infections.

The portal device 100 is comprised of catheter connector 102 carrying a distally projecting flexible tube 104 which defines a central lumen. The distal end 106 of the tube 104 is preferably beveled at 107 to facilitate insertion into the passageway proximal end of sleeve 30. The tube 104 preferably carries a conventional pinch clamp 108. The proximal end of connector 102 is preferably provided with a detachable fitting 110, e.g., Luer, for coupling to a hemostatic valve device 120, e.g., Touhy-Borst, of the type depicted in FIG. 9.

A Touhy-Borst hemostatic valve device 120 is well known in the art and readily commercially available, e.g., Qosina P/N 80375. It comprises a tube 122 defining a main lumen having an entrance port 124 and exit port 126. The distal end of tube 122 is formed with a Luer fitting 128 for coupling to the fitting 110 on the proximal end of portal device connector 102. An adjustable valve mechanism 126 is mounted near the proximal end of device 120 mounted between the main lumen entrance port 124 and exit port 126. The device 120 also includes a side arm 128 defining a side lumen having an entrance port 130 and an exit port 132 which opens into the main lumen upstream from exit port 126.

In general use, an implanted catheter is first extracted from sleeve 30 and then the projecting tube 104 of portal device 100 is inserted into the proximal end of the sleeve passageway 39. Depth markings 134 on the exterior surface of tube 104 assist the physician in properly inserting tube 104 into the sleeve passageway. With the portal device 100 properly installed to the sleeve, the hemostatic valve device 120 can then be coupled to the portal device via respective Luer fittings 110 and 128.

By way of background, it is generally known that the long term implantation of a catheter causes an internal tunnel to form around the catheter. The tunnel is composed of a fibrin sheath, or eventually, an epithelialized capsule, which isolates the tunnel interior from the surrounding subcutaneous tissue. The tunnel can be used to infuse suitable medication and/or provide interventional tool access.

In a specific application of a portal device in accordance with the invention to treat infection, first assume that a catheter has been implanted using the catheter assembly 20 depicted in FIGS. 1-6. Further assume that the physician has reason to suspect an infection is developing along the patient's interior body path. The physician then may proceed as follows:

a) Cut the suture/wire on the locking member 33.

b) Temporarily remove the locking member 33 from the catheter

c) Retract the catheter through the sleeve 30.

d) Insert the portal device projecting tube into the proximal end of the sleeve passageway 39.

e) Lock the locking member 33 to hold the portal device.

f) With the portal device locked by locking member 33, inject liquid medication into the proximal end of the portal device, e.g., via the Touhy-Borst hemostatic valve assembly side arm 128, for passage to the interior tunnel and venotomy site.

g) After a suitable interval, e.g., 24-48 hours, aspirate the liquid medication from the tunnel with a syringe through the Touhy-Borst side arm 128

h) Cut the suture/wire on the locking member 33 and remove the portal device.

i) Thread a new catheter through the tunnel to the venotomy site;

j) Lock the locking member 33 and secure with suture/wire;

In a different exemplary application to perform an angiographic procedure, the physician may proceed as follows:

a) Advance a guidewire through the implanted catheter

b) Cut the suture/wire on the locking member 33.

c) Retract the catheter through the sleeve 30.

d) Insert the portal device projecting tube into the proximal end of the sleeve passageway 39.

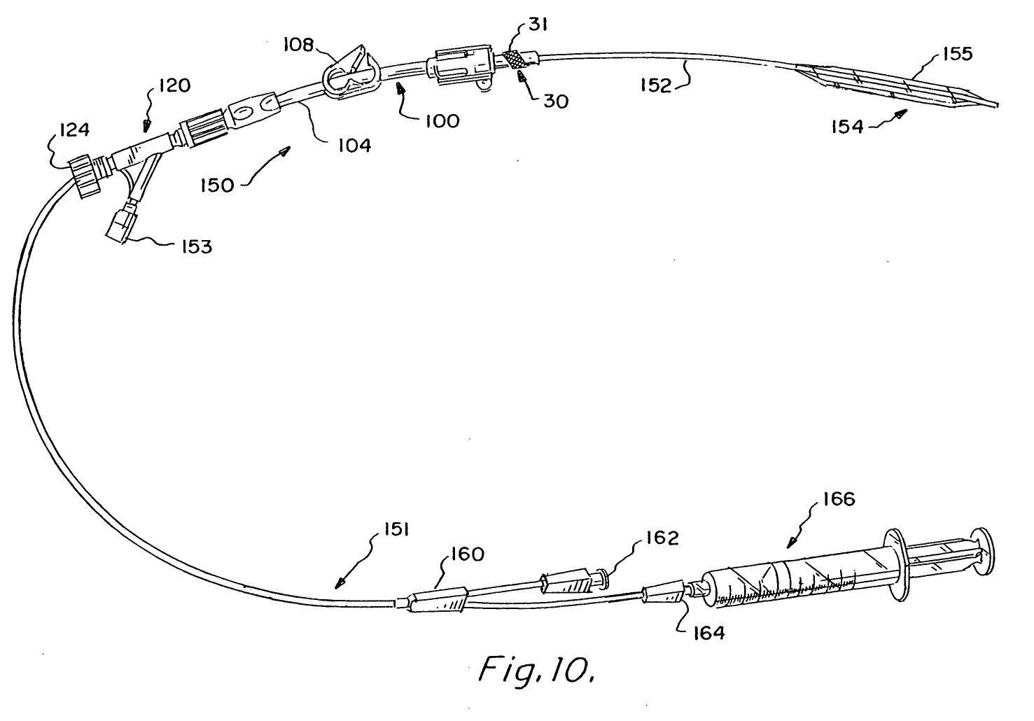

(e) Thread an angiographic catheter (FIG. 10) over the guide wire via the hemostatic valve assembly main entrance port 124.

(f) Inject radiopaque contrast dye via the valve side port 130 and visually observe with angiography.

(g) If a need for angioplasty is indicated, retract the angiography catheter and thread the angioplasty catheter onto the guide wire.

(h) Perform angioplasty to ablate the distal fibrin sheath.

(i) Retract the angioplasty catheter, the hemostatic valve assembly, and the portal device and thread a new hemodialysis catheter over the wire.

(j) Remove the wire.

FIG. 10 depicts an assembly 150 in accordance with the invention in combination with an exemplary tool, e.g., a balloon catheter 152, for performing an angioplasty procedure. The assembly 150 includes the aforedescribed sleeve 30 carrying porous layer 31, the portal device 100 including flexible tube 104, and the hemostatic valve device 120. FIG. 10 illustrates an exemplary balloon catheter 152 extending through the main lumen of the valve device 120, through the portal device 100 and through the sleeve 30 projecting past the sleeve distal end 42. The valve device side port 128 is shown closed by cap 153. The catheter 150 distal end 154 carries an expandable balloon 155. The proximal end 156 of catheter 152 extends proximally from the entrance port 124 of the valve device 120 and terminates in a conventional fitting 160 having dual entrance ports 162, 164. A syringe 166 is used to supply fluid via port 162 to the distal end 154 of catheter 150 to expand the balloon 155.

From the foregoing, it should now be understood that a method and apparatus have been described for facilitating the management of percutaneous conduits intended for long term implantation. Embodiments of the invention permit an implanted conduit to be withdrawn from the body through a percutaneous sleeve carrying porous material configured to integrate with a patient's dermal tissue. With the conduit removed, the interior tunnel can be used to pass procedural tools, e.g., an angiographic catheter, and/or to treat infections occurring at the incision site, a venotomy site, along the tunnel therebetween, or in the bloodstream.

Although, the preferred embodiment has been described with reference to a specific exemplary apparatus described in aforementioned application Ser. No. 11/708,445, it should be understood that the invention is also applicable to other structurally distinct, but functionally analogous, apparatus. Accordingly, it is recognized that various modifications and alternatives will occur to those skilled in the art consistent with the sprit of the invention and which fall within the intended scope of the appended claims.

1. A method of providing and maintaining long term access to an interior body site comprising:
forming an incision extending through a patient's epidermal and dermal layers;
providing a sleeve having a distal end, a proximal end, peripheral outer surface carrying a layer of porous material, and a peripheral inner surface defining a passageway;
providing an elongate conduit having a distal end and a proximal end and a peripheral outer surface dimensioned to fit within said sleeve passageway;
placing said distal end of said sleeve through said incision to position said porous material layer adjacent said patient's dermis layer for promoting tissue ingrowth;
axially sliding said conduit through said sleeve passageway;
selectively locking and unlocking said conduit to respectively prevent and permit relative movement between said conduit and said sleeve;
withdrawing said conduit from said sleeve;
introducing a tool into said sleeve passageway for performing an internal medical procedure;
withdrawing said tool; and
reinserting a new conduit into said sleeve passageway.

2. The method of claim 1 further including:
sealing a gap between said conduit outer surface and said sleeve inner surface.

3. The method of claim 1 further including:
mounting a hemostatic valve in series with said sleeve passageway.

4. The method of claim 1 further including:
selectively sealing said sleeve passageway.

5. A percutaneous conduit assembly including:
an elongate sleeve comprising a peripheral wall having an outer surface extending from a proximal end to a distal end and an inner surface surrounding an axially extending passageway;
an elongate conduit comprising a peripheral wall having an outer surface extending form a proximal end to a distal end;
said conduit extending through said sleeve passageway for axial slidable movement with respect thereto;
a sealing device in said sleeve for contacting said conduit outer surface to prevent migration of infectious material between said sleeve inner surface and said conduit outer surface;
a layer of porous material mounted on said sleeve outer surface adapted for subcutaneous placement in contact with a patient's dermal layer to promote tissue ingrowth and form an infection resistant barrier;
a locking member selectively operable in an unlocked state for permitting axial movement of said conduit through said sleeve passageway and in a locked state for preventing axial movement of said conduit through said sleeve passageway; and
a hemostatic valve mounted in series with said sleeve passageway configured to allow the insertion of elongate tools therethrough.

6. The assembly of claim 5 further including:
means for selectively sealing said sleeve passageway.

7. The assembly of claim 5 wherein said conduit comprises a catheter for transporting fluid.

8. The assembly of claim 5 further including a portal device comprising at least one lumen having a proximal end and a distal end;
said lumen distal end configured for detachable coupling to said sleeve passageway proximal end; and wherein
said hemostatic valve is mounted to said portal device in series with said lumen.
